(19) United States
(12) Patent Application Publication
(10) Pub. No.: US 2021/0190769 A1
(43) Pub. Date: Jun. 24, 2021

(54) DISTINGUISHING CANCER FROM NEUROLOGICAL RISK USING COPY NUMBER VARIATION

(71) Applicant: THE CLEVELAND CLINIC FOUNDATION, Cleveland, OH (US)

(72) Inventors: Charis Eng, Cleveland, OH (US); Dennis Lal, Cleveland, OH (US)

(21) Appl. No.: 17/128,446

(22) Filed: Dec. 21, 2020

Related U.S. Application Data
(60) Provisional application No. 62/951,518, filed on Dec. 20, 2019.

(51) Int. Cl. G01N 33/53

(52) U.S. Cl. CPC G01N 33/5308; G01N 2496/00; G01N 2800/50

(57) ABSTRACT

A method of providing a diagnosis for a subject having Cowden-like syndrome or PTEN germline mutations is described. The method includes the steps of: (a) obtaining a biological sample from a subject; (b) conducting a germline PTEN mutation and deletion analysis of genomic DNA from the biological sample; (c) determining the level of copy number variation in the genomic DNA; (d) comparing the level of copy number variation in the genomic DNA to a control value for copy number variation; and (e) diagnosing the subject as having an increased risk of developing a neurodevelopmental disorder if the copy number variation level is higher than the control value.

### CROSS-REFERENCE TO RELATED APPLICATIONS

This application claims priority to U.S. Provisional Patent Application No. 62/951,518, filed on Dec. 20, 2019, which is hereby incorporated by reference in its entirety.

### GOVERNMENT FUNDING

This invention was made with government support under Grant Nos. CA118989, CA124570, and NS092090 awarded by the National Institutes of Health. The U.S. Government has certain rights in this invention.

### BACKGROUND

The triumph of using genetics in precision medicine is the ability to stratify individuals at very high risk of disease and thereby enact specific gene-informed medical management. Topol E J, Cell., 157(1):241-253 (2014). In the case of heritable disease, genetics also allow testing of family members to determine with 100% accuracy whether they are at risk of disease. However, for any single individual carrying a mutation, the risk can be estimated by probabilities but not with certainty. For example, a germline mutation in gene X may give an 80% lifetime risk of disease Y, but for the single patient with that germline mutation, the risk of developing disease Y will be 100% or 0%. Searching for genomic modifiers of heritable disease risk at the individual level in humans has proven challenging. This makes estimation of clinical outcomes difficult, especially for phenotypically variable disorders. As such, why a mutation in one particular gene predisposes to disparate clinical outcomes, including in patients with identical mutations, remains poorly understood.

PTEN germline mutations include mutations of the phosphatase and tensin homolog (PTEN) gene (UCSC ID, uc001kfb.2; RefSeq, NM_000314), encoding deletions on chromosome 10. PTEN germline mutations are among the most common causes of autism spectrum disorder (ASD), accounting for approximately 2% to 5% of cases (Butler et al., J Med Genet., 42(4): 318-321 (2005)), yet originally identified in a subset of relatively rare syndromes predisposing to breast, thyroid, and other cancers. Liaw et al., Nat Genet., 16(1):64-67 (1997). The PTEN-related ASD-cancer phenotypic dichotomy and inability to preemptively estimate disease outcomes pose a challenge for more timely and precise clinical management. The concept of PTEN hamartoma tumor syndrome (PHTS) was proposed to encompass any clinical disorder with germline PTEN mutation on molecular genetic testing, regardless of phenotype. Marsh et al., Hum Mol Genet., 8(8):1461-1472 (1999). Based on this broad clinical spectrum and unified genetic etiology, PHTS serves as a useful disease model to identify modifiers of variable heritable disease risk at the individual level.

Copy number variation (CNV), a common type of structural variation in the human genome, is considered an important contributor to non-syndromic idiopathic ASD and sporadic cancer. Jeste S S, Geschwind D H., Nat Rev Neurol., 10(2):74-81 (2014); Huang et al; Cell, 173(2):355-370.e14 (2018). Copy number variations have also been associated with complex disorders, particularly those involving developmental delay, intellectual disabilities, and/or congenital anomalies. Miller et al., Am J Hum Genet., 86(5):749-764 (2010). However, it was not known whether specific CNV associations in patients carrying germline PTEN mutations are associated with development of specific clinical phenotypes at an individual level.

### SUMMARY OF THE INVENTION

The present invention provides a method of providing a diagnosis for a subject having Cowden-like syndrome or PTEN germline mutations. The method includes the steps of: (a) obtaining a biological sample from a subject; (b) conducting a germline PTEN mutation and deletion analysis of genomic DNA from the biological sample; (c) determining the level of copy number variation in the genomic DNA; (d) comparing the level of copy number variation in the genomic DNA to a control value for copy number variation; and (e) diagnosing the subject as having an increased risk of developing a neurodevelopmental disorder if the copy number variation level is higher than the control value. In some embodiments, a copy number variation level that is higher than the control value indicates a higher risk of developing a neurodevelopmental disorder than the risk of developing cancer.

In some embodiments, the neurodevelopmental disorder is autism spectrum disorder. In further embodiments, the genomic DNA comprises the PTEN promoter region. In yet further embodiments, the deletion analysis is conducted using a multiplex ligation-dependent probe amplification assay.

In some embodiments, the mutation analysis comprises denaturing gradient gel electrophoresis, high-resolution melting curve analysis, and directed Sanger sequencing. In additional embodiments, the step of determining the level of copy number variation comprises single-nucleotide polymorphism genotype quality control.

In some embodiments, the subject has Cowden-like syndrome. In further embodiments, the subject has PTEN germline mutations. In yet further embodiments, the germline PTEN mutations are pathogenic germline PTEN mutations.

The inventors conducted a prospective cohort study that examined genome-wide microarrays performed on blood-derived DNA to detect germline CNVs from participants, including patients with PTEN hamartoma tumor syndrome (PHTS), molecularly defined as carrying germline pathogenic PTEN mutations. The prevalence of pathogenic and/or likely pathogenic CNVs in patients with PHTS and association with ASD/developmental delay and/or cancer, ascertained through medical records and pathology reports.

The study included 481 patients with PHTS (mean [SD] age, 33.2 [21.6] years; of which 268 were female [55.7%]). The analytic series consisted of 309 patients with PHTS and genetically determined European ancestry. Patients were divided into 3 phenotypic groups, excluding family members within each group. These include 110 patients with ASD/developmental delay, 194 without ASD/developmental delay, and 121 with cancer (of whom 116 were in the no ASD/developmental delay group).

Genome-wide evaluation of autosomal CNVs indicated an increased CNV burden, particularly duplications in genic regions, in patients with ASD/developmental delay compared with those without ASD/developmental delay (odds ratio [OR], 1.9; 95% CI, 1.1-3.4; P=0.03) and those with cancer (OR, 2.5; 95% CI, 1.3-4.6; P=0.003). Eleven of the 110 patients (10.0%) with ASD/developmental delay carried pathogenic and/or likely pathogenic CNVs associated with neurodevelopmental disorders, compared with 5 of 194 (2.6%) without ASD/developmental delay (OR, 4.2; 95% CI, 1.4-13.7; P=0.008) and 2 of 121 (1.7%) with cancer (OR, 6.6; 95% CI, 1.6-44.5; P=0.007). Evidence of an association between pathogenic and/or likely pathogenic CNVs and PHTS with ASD/developmental delay was further supported in a validation series of 69 patients with PHTS of genetically determined non-European ancestry. These findings indicate that copy number variations are associated with the ASD/developmental delay clinical phenotype in PHTS, providing proof of principle for similarly heterogeneous disorders lacking outcome-specific associations.

### BRIEF DESCRIPTION OF THE FIGURES

The present invention may be more readily understood by reference to the following figures, wherein:

FIGS. 1A and 1B provide graphs showing (A) Genome-wide CNV burden representing the total number of CNVs in each patient within the 3 PHTS phenotype groups. The line in the middle of each box is plotted at the median. Each box extends from the 25th to 75th percentiles (interquartile range [IQR]). Whiskers and outliers (circles) are plotted according to the Tukey method. The upper whiskers extend to the largest value no further than 1.5 times the IQR. The lower whiskers extend to the smallest value no further than 1.5 times the IQR; (B) Stratified analyses showing CNV burden (duplications and deletions) in genic and nongenic genomic regions, accounted for by the number of unrelated patients carrying such CNVs within each clinical phenotype group. The size of each box is proportional to the respective odds ratio (OR) depicted in the adjacent table. ASD indicates autism spectrum disorder; DD, developmental delay.

FIG. 2 provides a graph showing pathogenic and/or likely pathogenic CNVs associated with neurodevelopmental disorders were found in 11 of 110 patients in the ASD/DD group (10.0%), 5 of 194 patients in the no ASD/DD group (2.6%), and 2 of 121 patients in the subgroup with cancer (1.7%). The size of each box is proportional to the respective odds ratio (OR) depicted in the adjacent table.

FIGS. 3A-3C provides graphs showing PTEN mutation spectra across the PHTS Clinical Phenotype Groups. PTEN mutations are depicted in the lollipop plots overlying the PTEN protein structures. The frequency of each mutation correlates with the heights of the vertical lines representing each lollipop. A, PTEN mutations identified in PHTS individuals with autism spectrum disorder/developmental delay (ASD/DD). Three whole PTEN gene deletions, one exon 1 deletion, and one exon 1 duplication are not depicted. B, PTEN mutations identified in PHTS individuals without ASD/DD, including the subset with cancer. Two whole PTEN gene deletions, three exon 1 deletions, two exon 1-2 deletions, one exon 6 duplication, one exon 2-9 duplication, and 21 promoter mutations are not depicted. C, PTEN mutations identified in PHTS individuals with cancer. One whole PTEN gene deletion, one exon 1 deletion, one exon 1-2 deletions, one exon 6 duplication, and 20 promoter mutations are not depicted. The cBioPortal MutationMapper tool was used to depict the identified PTEN germline mutations. Family members were excluded from each PHTS phenotype group. The ‘other’ category includes the PTEN p.M11 start loss mutation.

### DETAILED DESCRIPTION OF THE INVENTION

The present invention provides a method of providing a diagnosis for a subject having Cowden-like syndrome or PTEN germline mutations. The method includes the steps of: (a) obtaining a biological sample from a subject; (b) conducting a germline PTEN mutation and deletion analysis of genomic DNA from the biological sample; (c) determining the level of copy number variation in the genomic DNA; (d) comparing the level of copy number variation in the genomic DNA to a control value for copy number variation; and (e) diagnosing the subject as having an increased risk of developing a neurodevelopmental disorder if the copy number variation level is higher than the control value.

### Definitions

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

The term “gene” as used herein refers to a nucleotide sequence that can direct synthesis of an enzyme or other polypeptide molecule (e.g., can comprise coding sequences, for example, a contiguous open reading frame (ORF) which encodes a polypeptide) or can itself be functional in the organism. A gene in an organism can be clustered within an operon, as defined herein, wherein the operon is separated from other genes and/or operons by intergenic DNA. Individual genes contained within an operon can overlap without intergenic DNA between the individual genes.

The term “genotype” refers to the alleles present in DNA from a subject or patient, where an allele can be defined by the particular nucleotide(s) present in a nucleic acid sequence at a particular site(s). Often a genotype is the nucleotide(s) present at a single polymorphic site known to vary in the human population.

A “germline mutation,” as used herein, refers to a heritable change in the DNA that occurred in a germ cell (a cell destined to become an egg or in the sperm) or the zygote (the conceptus) at the single-cell stage. When transmitted to a child, a germline mutation is incorporated in every cell of their body.

“Copy number variation,” as used herein, revers to a number of genes that is different from the norm (which is typically two copies). Variation in the copy number is generally caused by deletion or duplication of genes.

As used herein, the term “polymorphism” refers to a difference in the nucleotide sequence of a given region as compared to a nucleotide sequence in a homologous region of another individual, in particular, a difference in the nucleotide sequence of a given region that differs between individuals of the same species. Polymorphisms include single nucleotide differences, differences in sequence of more than one nucleotide, insertions, inversions and deletions. The term refers to the occurrence of two or more genetically determined alternative sequences or alleles in a population. A polymorphic marker is the locus at which divergence occurs. Preferred markers have at least two alleles, each occurring at frequency of greater than 1%. A polymorphic locus can be as small as one base pair.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein and in the appended claims, the singular forms “a”, “and”, and “the” include plural referents unless the context clearly dictates otherwise. Thus, for example, reference to “a sample” also includes a plurality of such samples.

Unless defined otherwise, all technical and scientific terms used herein have the same meaning as commonly understood by one of ordinary skill in the art to which this invention belongs.

### Diagnosing Neurodevelopmental Disorder

One aspect of the invention provides a method of providing a diagnosis for a subject having Cowden-like syndrome or PTEN germline mutations, the method comprising the steps of: (a) obtaining a biological sample from a subject; (b) conducting a germline PTEN mutation and deletion analysis of genomic DNA from the biological sample; (c) determining the level of copy number variation in the genomic DNA; (d) comparing the level of copy number variation in the genomic DNA to a control value for copy number variation; and (e) diagnosing the subject as having an increased risk of developing a neurodevelopmental disorder if the copy number variation level is higher than the control value. In some embodiments, a copy number variation level that is higher than the control value indicates a higher risk of developing a neurodevelopmental disorder than the risk of developing cancer.

### Neurodevelopmental Disorders

The present invention provides methods for diagnosing subjects having an increased risk of developing a neurodevelopmental disorder. Neurodevelopmental disorders are disabilities associated primarily with the functioning of the neurological system and brain. Examples of neurodevelopmental disorders in children include attention-deficit/hyperactivity disorder (ADHD), autism, learning disabilities, intellectual disability (also known as mental retardation), conduct disorders, cerebral palsy, and impairments in vision and hearing. Learning disability (or learning disorder) is a general term for a neurological disorder that affects the way in which a child's brain can receive, process, retain, and respond to information. Children with neurodevelopmental disorders can experience difficulties with language and speech, motor skills, behavior, memory, learning, or other neurological functions.

In some embodiments, the neurodevelopmental disorder is autism spectrum disorder. Autism spectrum disorders (ASDs) are a group of developmental disabilities defined by significant social, communication, and behavioral impairments. The term “spectrum disorders” refers to the fact that although people with ASDs share some common symptoms, ASDs affect different people in different ways, with some experiencing very mild symptoms and others experiencing severe symptoms. ASDs encompass autistic disorder and the generally less severe forms, Asperger's syndrome and pervasive developmental disorder-not otherwise specified (PDD-NOS). Children with ASDs may lack interest in other people, have trouble showing or talking about feelings, and avoid or resist physical contact. A range of communication problems are seen in children with ASDs: some speak very well, while many children with an ASD do not speak at all. Another hallmark characteristic of ASDs is the demonstration of restrictive or repetitive interests or behaviors, such as lining up toys, flapping hands, rocking his or her body, or spinning in circles. Examples of ASDs are autistic disorder, Asperger's disorder, and pervasive developmental disorder. McPartland, J., and Volkmar, F., Handb Clin Neurol, 106:407-18 (2012).

### Biological Samples

The method of diagnosis includes the step of obtaining a biological sample from a subject. A “biological sample,” as used herein, is meant to include any biological sample from a subject that is suitable for detection and analysis of the copy number variation in the genomic DNA of the subject. Suitable biological samples include but are not limited to bodily fluids such as blood-related samples (e.g., whole blood, serum, plasma, and other blood-derived samples), urine, sputem, cerebral spinal fluid, bronchoalveolar lavage, and the like. Another example of a biological sample is a tissue sample. In some embodiments, the biological sample is a cancer cell or tissue including cancer cells. The copy number variation in the genomic DNA of the subject can be assessed either quantitatively or qualitatively, and detection can be conducted either in vitro or ex vivo.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a tissue sample expressly obtained for the assays of this invention or a tissue sample obtained for another purpose which can be subsampled for the assays of this invention. Preferably, biological samples are either chilled or frozen shortly after collection if they are being stored to prevent deterioration of the sample.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC) or HPLC, or precipitation of apolipoprotein B containing proteins with dextran sulfate or other methods. Any of a number of standard aqueous buffer solutions at physiological pH, such as phosphate, Tris, or the like, can be used.

### Subjects

The terms “individual,” “subject,” and “patient” are used interchangeably herein irrespective of whether the subject has or is currently undergoing any form of treatment. As used herein, the term “subject” generally refers to any vertebrate, including, but not limited to a mammal Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment or diagnosis of humans is of particular interest.

In some embodiments, the subject has Cowden syndrome or Cowden-like syndrome. Cowden syndrome (also known as Cowden's disease and multiple hamartoma syndrome) is an autosomal dominant inherited condition characterized by benign overgrowths called hamartomas as well as an increased lifetime risk of breast, thyroid, uterine, and other cancers. It is often underdiagnosed due to variability in disease presentation, but 99% of patients report mucocutaneous symptoms by age 20-29. Cowden's syndrome is a multi-system disorder that also includes neurodevelopmental disorders such as macrocephaly.

Cowden syndrome is a great clinical mimic and can be difficult to recognize because every patient shows variable expression and penetrance. Many individuals in the general population share one or more features of Cowden syndrome but may not have Cowden syndrome and may not even harbor alterations in any predisposition genes. The presentation of Cowden syndrome is strikingly diverse; some patients exhibit characteristic features without meeting diagnostic criteria for CS. Cowden-like syndrome (CLS) is the diagnosis in these patients, few of whom have mutations in PTEN or variants in the succinate dehydrogenase (SDHB) gene.

Only 5% of the heterogeneous group having Cowden-like syndrome and who have some features of Cowden syndrome but do not meet diagnostic criteria, have germline PTEN mutations. In the absence of germline PTEN mutations, approximately 10% of individuals with Cowden syndrome or Cowden-like syndrome harbor germline succinate dehydrogenase variants SDHB and SDHD. Overall, germline PTEN mutations and deletions and SDHx variants account for 35% of Cowden syndrome and 6% to 11% of individuals with Cowden-like syndrome phenotypic features.

In some embodiments, the subject has germline PTEN mutations. Cowden syndrome is associated with mutations in the phosphatase and tensin homolog (PTEN) gene (UCSC ID, uc001kfb.2; RefSeq, NM_000314) on 10q23.3, a tumor suppressor gene otherwise known as phosphatase and tensin homolog, that results in dysregulation of the mTOR pathway leading to errors in cell proliferation, cell cycling, and apoptosis. Germline mutations of the PTEN gene, encoding deletions on chromosome 10, cause 25% of autosomal-dominant Cowden syndrome which minimally occur in 1 in 200,000 live births. These mutations result in a syndrome characterized by macrocephaly and typical mucocutaneous features (trichilemmomas, papillomatous papules) and hamartomas, with increased risk of various malignancies, approximately 28% lifetime risk for thyroid cancer, and as much as 50% lifetime risk for female breast cancer over the general population.

In some embodiments, the germline PTEN mutations are pathogenic germline PTEN mutations. Pathogenicity predictions were ascertained by reports from orthogonal testing in a Clinical Laboratory Improvement Amendments of 1988—certified facility, ClinVar database classifications, and/or the ClinGen gene-specific criteria for PTEN variant curation. Mester et al., Hum Mutat., 39(11):1581-1592 (2018).

In some embodiments, the method is used to determine whether the subject has a higher risk of developing a neurodevelopmental disorder than the risk of developing cancer. As used herein, the terms “tumor” or “cancer” refer to a condition characterized by anomalous rapid proliferation of abnormal cells of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize.

### Determining the Level of Copy Number Variation in the Genomic DNA

The method of diagnosis includes the step of conducting a germline PTEN mutation and deletion analysis of genomic DNA from the biological sample. In some embodiments the genomic DNA comprises the PTEN region, while in further embodiments the DNA comprises the PTEN promoter region. “Mutation and deletion analysis” is defined herein as the study or analysis of DNA fragments to determine if the fragments contain variations (i.e., mutations or deletions) in a population.

Methods of carrying out mutation analysis of DNA are known by those skilled in the art. Mutations on germline or somatic DNA include mis-sense and nonsense mutations, SNPs (single nucleotide polymorphisms) deletions and insertions. In some embodiments, the mutation analysis comprises denaturing gradient gel electrophoresis, high-resolution melting curve analysis, and directed Sanger sequencing. Other types of mutation analysis include single-strand conformation polymorphism (SSCP) analysis, SSCP/heteroduplex analysis, enzyme mismatch cleavage, allele-specific hybridization, and restriction analysis of the genomic DNA.

In some embodiments, the mutation analysis is specifically interested in detecting deletions, in which case it is referred to as deletion analysis. Methods of conducting deletion analysis are also known to those skilled in the art. In some embodiments, the deletion analysis is conducted using a multiplex ligation-dependent probe amplification assay.

Many methods of mutation analysis involve PCR. As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. Examples of methods for determining a nucleic sequence involving PCR include quantitative RT-PCR, Northern blot, real-time PCR, PCR, allele-specific PCR, pyrosequencing, SNP Chip technology, and restriction fragment length polymorphism (RFLP).

In some embodiments, hybridization with complementary sequences may be used to detect the presence of germline PTEN mutations based on the different characteristics of sequences that have a complete or incomplete sequence match. For example, an asymmetric PCR assay can be used in which fluorescence melting reveals two distinct melting temperatures of the probe/target duplex that are specific for the amplified sequence.

In some embodiments, the method comprises single-nucleotide polymorphism genotype quality control, in order to correct for population stratification. For example, in some embodiments, DNA quality can be evaluated using spectrophotometry. Genotype quality control can include the use of principal component analysis to assess population stratification. Quality control commonly includes algorithmic processing of the nucleotide sequence data, such as intensity-based quality control to exclude samples with low quality based on waviness factor.

The method of diagnosis also includes the step of determining the level of copy number variation in the genomic DNA. Copy number variation is a type of structural variation where you have a stretch of DNA, which can be duplicated, or sometimes even triplicated or quadruplicated. Therefore, when you look at that chromosomal region, you will see a variation in the number of copies. Sometimes those copy number variants include genes, maybe several genes, which may mean that an individual has four copies of that gene instead of the usual two, while a different individual has three, and a further individual has five.

Methods of determining copy number variation are known to those skilled in the art. See, for example, U.S. patent application Ser. No. 16/913,965; Hastings et al., Nat Rev Genet; 10(8):551-64 (2009); and Shishido et al., Psychiatry Clin Neurosci, 68(2):85-95 (2014), the disclosures of which are incorporated by reference herein. For example, in some embodiments, determining copy number variation includes the steps of: a. providing at least two sets of first polynucleotides, wherein each set maps to a different reference sequence in a genome, and, for each set of first polynucleotides; i. amplifying the polynucleotides to produce a set of amplified polynucleotides; ii. sequencing a subset of the set of amplified polynucleotides, to produce a set of sequencing reads; iii. grouping sequences reads sequenced from amplified polynucleotides into families, each family amplified from the same first polynucleotide in the set; iv. inferring a quantitative measure of families in the set; v. determining copy number variation by comparing the quantitative measure of families in each set.

After determining the level of copy number variation in the genomic DNA, the level of copy number variation in the genomic DNA is compared to a control value to determine the relative level of copy number variation. The control value can be the level of copy number variation in subjects having Cowden-like syndrome or PTEN germline mutations. Alternately, the control value can be the level of copy number variation in subjects having Cowden-like syndrome or PTEN germline mutations who have been diagnosed with cancer.

Once the presence and/or levels of copy number variation have been determined, they can be displayed in a variety of ways. For example, the levels can be displayed graphically on a display as numeric values or proportional bars (i.e., a bar graph) or any other display method known to those skilled in the art. The graphic display can provide a visual representation of the amount of copy number variation in the biological sample being evaluated.

A subject can be diagnosed as having an increased risk of developing a neurodevelopmental disorder if the copy number variation level is higher than the control value. As shown in FIG. 2, patients with autism spectrum disorder (ASD) or developmental delay (DD) show higher levels of copy number variation. For example, in some embodiments, cancer patients having Cowden-like syndrome or PTEN germline mutations may show a level of copy number variation of about 1%-2%, while patients having a neurodevelopmental disorder such as ASD or DD may show a level of copy number variation of about 3%-15%, 5%-15%, 8%-12%, or about 10%. Note that subjects that have neither cancer nor a neurodevelopmental disorder may show a level of copy number variation of from about 2% to about 3%.

### Therapeutic Methods

Another aspect of the invention provides a method of treating a subject who has been diagnosed as having or having an increased risk of having a neurodevelopmental disorder. The specific type of therapy provided depends on the particular type of neurodevelopmental disorder, though generally the therapy involved physical or behavioral therapy. For example, see Lucas et al., BMC Pediatr, 29; 16(1):193 (2016), which describes interventions to improve gross motor performance in children with neurodevelopmental disorders. However, drugs such as glutamate receptor 5 antagonists and GABA_{B} receptor agonists can also be used to treat neurodevelopmental disorders. Berry-Kravis et al., Nat Rev Drug Discov, 17(4):280-299 (2018). A preferred method for treating ASD is applied heavier analysis (ABA) therapy. Fernandes et al., Codas, 25(3):289-96 (2013).

An example has been included to more clearly describe a particular embodiment of the invention. However, there are a wide variety of other embodiments within the scope of the present invention, which should not be limited to the particular example provided herein.

### EXAMPLE

### Copy Number Variation and Clinical Outcomes in Patients with Germline PTEN Mutations

PTEN is among the most common autism spectrum disorder (ASD)-predisposition genes. Germline PTEN mutation carriers can develop malignant neoplasms and/or neurodevelopmental disorders such as ASD and developmental delay. Why a single gene contributes to disparate clinical outcomes, even in patients with identical PTEN mutations, was unclear. The objective of this work was therefore to investigate the association of copy number variations (CNVs), altered numbers of copies of DNA sequences within the genome, with specific phenotypes in patients with germline PTEN mutations.

### Methods

### Patients

A total of 6782 patients were prospectively accrued from Sep. 1, 2005, through Jan. 3, 2018, and provided informed written consent to participate. This prospective cohort study was approved by the Cleveland Clinic institutional review board. This study followed the Strengthening the Reporting of Observational Studies in Epidemiology (STROBE) reporting guideline for cohort studies. Inclusion criteria for enrollment included meeting at least the relaxed International Cowden Consortium operational diagnostic criteria, meaning full diagnostic criteria minus 1 feature, termed Cowden-like syndrome (Eng C., J Med Genet., 37(11): 828-830 (2000)); having macrocephaly plus neurodevelopmental disorders (e.g, ASD, developmental delay [DD], mental retardation) and/or penile freckling; or the presence of a known pathogenic germline PTEN mutation (patients referred to the PTEN Multidisciplinary Clinic at the Cleveland Clinic). These patients were broadly recruited from community and academic medical centers throughout North America, South America, Europe, Australia, and Asia, as noted by Tan et al. Tan et al., Clin Cancer Res. 2012; 18(2):400-407 (2012). After informed consent was obtained, checklists to document the presence or absence of specific features were completed by specialist genetic counselors or physicians concurrently with withdrawal of blood specimen (team led by C.E., medical director, PTEN/Cowden Multidisciplinary Clinic, Center for Personalized Genetic Healthcare, Genomic Medicine Institute, Cleveland Clinic, Cleveland, Ohio). Specialist genetics staff reviewed all checklists and corresponded with the enrolling center if necessary to obtain further primary documentation of medical records and, for cancer, pathology reports, for phenotype confirmation with patient consent. Tan et al., Am J Hum Genet. 2011; 88(1):42-56 (2011). For each consenting patient, the inventors reviewed medical records, including pedigrees, clinical genetic testing reports, and clinical notes associated with genetics evaluations, ASD Diagnostic and Statistical Manual of Mental Disorders (Fourth Edition, Text Revision) criteria, and/or genetic counseling visits, where applicable. The inventors followed up this cohort prospectively for development of phenotypic features.

### PTEN Mutation and Deletion Analysis

All eligible patients underwent germline PTEN mutation and deletion analysis. Genomic DNA was extracted from peripheral blood samples using standard protocols. Mutation analysis was standardly performed, with a combination of denaturing gradient gel electrophoresis, high-resolution melting curve analysis (Idaho Technology), and direct Sanger sequencing (ABI 3730xl; Applied Biosystems, Life Technologies). Deletion analysis using the multiplex ligation-dependent probe amplification assay was performed with the multiplex ligation-dependent probe amplification kit (P158; MRC-Holland) according to manufacturer protocol. Ngeow et al., J Clin Oncol., 32(17):1818-1824 (2014). All patients underwent polymerase chain reaction-based Sanger sequencing of the PTEN promoter region. Only cases with pathogenic or likely pathogenic germline PTEN mutations were included in the analytic sample. To be conservative, individuals with PTEN promoter variants were considered mutation positive only if the underlying variants had been previously associated with PHTS or known to affect PTEN function. Teresi et al., Am J Hum Genet., 81(4):756-767 (2007); Wang et al., Oncogene, 30(42):4327-4338 (2011). Pathogenicity predictions were ascertained by reports from orthogonal testing in a Clinical Laboratory Improvement Amendments of 1988—certified facility, ClinVar database classifications, and/or the ClinGen gene-specific criteria for PTEN variant curation. Mester et al., Hum Mutat., 39(11):1581-1592 (2018). The resultant data set represents the largest deeply phenotyped worldwide series of patients with PHTS from the International Cowden Consortium.

### Genotyping, CNV Calling, and Quality Control

DNA quality (A260/A280) was evaluated using spectrophotometry (NanoDrop 1000; Thermo Fisher Scientific) and quantity using a double-stranded DNA high-sensitivity assay kit (Qubit; Thermo Fisher Scientific). DNA samples were all genotyped on a commercially available genotyping array (Infinium Global Screening Array-24, version 1.0; Illumina) at the Broad Institute Genomic Services, Cambridge, Mass. A total of 642 824 markers were used for quality control. To correct for population stratification, single-nucleotide polymorphism (SNP) genotype quality control was performed. Samples with a call rate of less than 0.91 or with discordant sex were excluded. The inventors focused on autosomal CNVs owing to higher fidelity of CNV calling compared with sex chromosomes. Pinto et al., Nat Biotechnol., 29(6):512-520 (2011). To perform principal component analysis to assess population stratification, autosomal SNPs with low genotyping rates (>0.98) and deviation from Hardy-Weinberg equilibrium (P<0.001) were filtered before pruning SNPs for linkage disequilibrium using default parameters in PLINK, version 1.927 (--indep-pairwise 200 100 0.2). Chang et al., Gigascience, 4:7 (2015). The PennCNV algorithm (Wang et al. Genome Res., 17(11):1665-1674 (2007)) was used to generate GC DNA nucleotide wave-adjusted log R ratio intensity files and to call CNVs in all samples. A custom population B-allele frequency file was generated before CNV calling. Adjacent CNV calls were merged if the number of intervening markers was less than 20% of the total number than when both segments were combined. Next, the inventors performed intensity-based quality control to exclude samples with low quality based on waviness factor, log R ratio SD, and B-allele frequency drift. These thresholds were calculated by taking the median plus 3 SD to identify outliers. Huang et al; Neuron., 94(6):1101-1111 (2017). After intensity-based quality control, all samples had a log R ratio SD of less than 0.25, an absolute waviness factor of less than 0.04, and a B-allele frequency drift of less than 0.007. Samples with an excessively high CNV load (>100) as determined empirically by visual inspection of CNV distribution across a combined set of samples were excluded. None of the PHTS samples had a CNV load above this threshold (with a maximum of 16 CNVs detected in 1 patient). Finally, CNVs spanning less than 20 markers, CNVs of less than 20 Kb in length, CNVs with SNP density (number of markers/CNV length) of less than 0.0001, and/or CNVs with greater than 50% of total length overlapping artifactual regions in SNP-based CNV calling were excluded. Marshall et al., Nat. Genet. 49(1):27-35 (2017). The final data set consisted of 309 patients of genotype-determined European ancestry with at least 1 CNV call.

### Genome-Wide CNV Burden Analysis

The CNV burden among patients with PHTS and ASD and/or developmental delay (ASD/DD group), those without ASD or developmental delay (no ASD/DD group), and those with cancer was measured. Genome-wide burden was also analyzed for only rare CNVs (excluding CNVs overlapping >80% of regions known to be recurrent in the general population). Duplications and deletions were analyzed in combination as well as separately. Moreover, within each category (duplications and deletions), genic and nongenic CNVs were analyzed in combination as well as separately. For all comparisons, the number of unrelated probands with such genomic events was counted within each phenotype group.

### CNV Annotation and Identification of Clinically Relevant CNVs

A CNV was considered as genic if it overlapped more than 80% of a gene (Coppola et al., Epilepsia, 60(4), 689-706 (2019)); otherwise it was annotated as nongenic. Cancer-relevant loci included 46 genes associated with Cowden syndrome component cancers, of which 24 are clinically actionable cancer-related genes according to the American College of Medical Genetics and Genomics guidelines. Yehia et al., PLoS Genet., 14(4):e1007352 (2018). Genes associated with ASD were curated from the Simons Foundation Autism Research Initiative Gene module.32 Only syndromic (category S), high-confidence (category 1), and strong candidate (category 2) genes were retained. The inventors also interrogated regions associated with genomic disorders, congenital malformations, and neurodevelopmental phenotypes, including 48 loci from DECIPHER (Database of Chromosomal Imbalance and Phenotype Using Ensembl Resources), 298 genes from the Developmental Disorders Genotype-Phenotype Database, and 92 loci of pathogenic CNVs from the UK Biobank. Pathogenicity and likely pathogenicity of previously unreported CNVs were estimated based on size and gene content according to the American College of Medical Genetics and Genomics guidelines. Human Phenotype Ontology terms were used for standard annotation of CNVs with associated phenotypic abnormalities.

### Statistical Analysis

Data were analyzed from Nov. 14, 2018, to Aug. 1, 2019. Patient demographic characteristics were reported by age, sex, and clinical phenotypes. OpenEpi software was used to calculate odds ratios (ORs) for burden and enrichment analyses. For analyses among different patient phenotype groups, 2×2 tables were used to calculate ORs. The 95% CIs and corresponding P values were calculated using the mid-P exact test. The nonparametric Mann-Whitney test was used to analyze CNV burden per individual among the different patient phenotype groups. All P values were 2-sided and considered to be significant at P<0.05. We used Prism 8, version 8.1.1 (GraphPad) to generate box-and-whisker and forest plots.

### Results

### Patient Characteristics and PTEN Mutation Spectrum

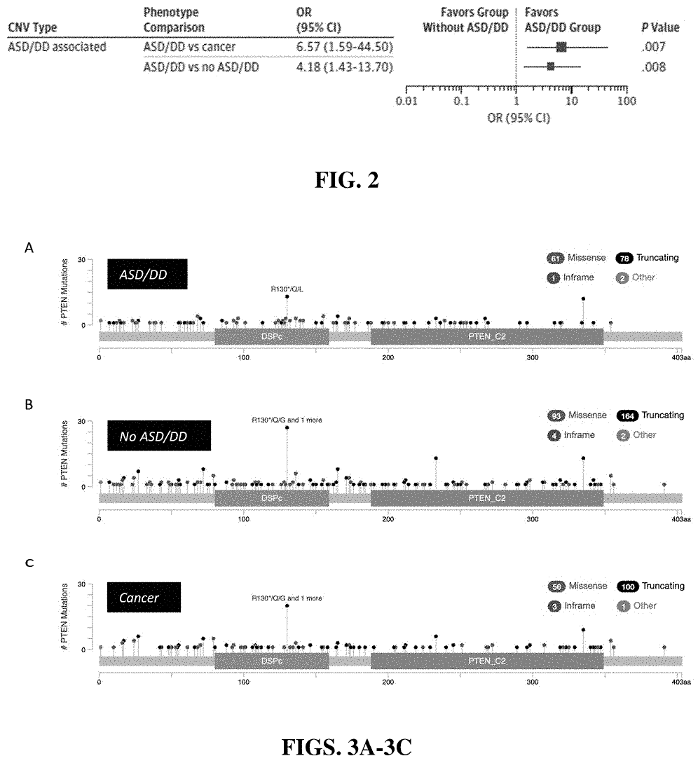

At baseline, the inventors prospectively accrued 481 patients with PHTS (mean [SD] age at consent, 33.2 [21.6] years; 268 female [55.7%] and 213 male [44.3%]). The Cleveland Clinic score, a quantitative surrogate of age-related PHTS disease burden, ranged from 0 to 69 (mean [SD], 20 [13]), corroborating the broad phenotypic spectrum, including participants with unexpectedly mild disease manifestations (e.g., absence of early-onset component cancers and neurotypical development) despite their underlying pathogenic germline PTEN mutations. Patients with PHTS were stratified into 3 phenotypically ascertained groups, including the ASD/DD group, no ASD/DD group, and cancer group (a subset of the no ASD/DD group). They did not include a no cancer group owing to the inability to ascertain cancer diagnoses, particularly in pediatric, adolescent, and young adult patients. Importantly, the PTEN mutation spectra were similar across the 3 PHTS phenotype groups (FIGS. 3A-3C).

Genome-Wide CNV Burden in Patients with PHTS

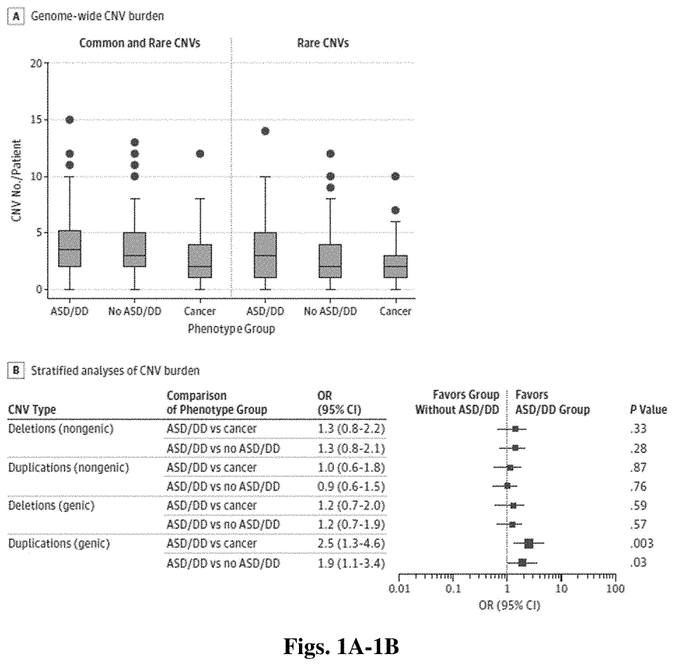

The analytic sample consisted of 309 patients with PHTS (Table 1) of genetically determined European ancestry (mean [SD] age at consent, 32.0 [21.0] years; 167 female [54.0%] and 142 male [46.0%]). Phenotypically ascertained groups consisted of 110 unrelated patients in the ASD/DD phenotype group, 194 unrelated patients in the no ASD/DD phenotype group, and a subset of 121 unrelated patients, including 116 in the no ASD/DD group, with cancer. To determine the genomewide CNV burden, the total number of CNVs in each participant were quantified within the 3 PHTS phenotype groups (FIG. 1A). Patients in the ASD/DD group had an overall increased CNV burden per individual (median, 3.5; range, 0-15) compared with those in the no ASD/DD group (median, 3.0; range, 0-13; Mann-Whitney 2-sided test comparing ranks, P=0.04) and the subset with cancer (median, 2.0; range, 0-12; Mann-Whitney 2-sided test, P=0.002). Similarly, patients in the ASD/DD group had an overall increased burden of rare CNVs per individual (median, 3.0; range, 0-14) compared with those in the no ASD/DD group (median, 2.0; range, 0-12; Mann-Whitney 2-sided test comparing ranks, P=0.01) and the subset with cancer (median, 2.0; range, 0-10; Mann-Whitney 2-sided test, P<0.001). No difference was observed in CNV burden among patients in the no ASD/DD group (median, 3.0; range, 0-13) compared with the subset with cancer (median, 2.0; range, 0-12; Mann-Whitney 2-sided test, P=0.15). Similarly, no difference was observed in rare CNV burden among patients in the no ASD/DD group (median, 2.0; range, 0-12) compared with the subset with cancer (median, 2.0; range, 0-10; Mann-Whitney 2-sided test, P=0.19). Stratified analyses according to CNV type and genomic locus revealed that patients in the ASD/DD group had a significant enrichment of duplications in genic regions (FIG. 1B), compared with patients in the no ASD/DD group (OR, 1.9; 95% CI, 1.1-3.4; P=0.03) and the subset with cancer (OR, 2.5; 95% CI, 1.3-4.6; P=0.003).

TABLE 1
Clinical Characteristics of the Analytic Series of 309 Patients
With PHTS and European Ancestry

| Clinical Phenotypic Characteristics | Data |
|---|---|
| Sex, No. (%) | |
| Female | 167 (54.0) |
| Male | 142 (46.0) |
| Age at consent, mean (SD) [range], y | 32 (21) [1-85] |
| No. of germline PTEN mutations | |
| Promoter | 8 |
| Missense | 99 |
| Nonsense | 86 |
| Splice site | 28 |
| Frameshift truncating | 63 |
| Insetions and deletions | 25 |
| Neurodevelopment features, No. (%) | 110 (35.6) |
| Autism spectrum disorder | 45 (14.6) |
| Global development delay | 56 (18.1) |
| Variable delay | 25 (8.1) |
| Mental retardation | 9 (2.9) |
| Learning disabilities | 9 (2.9) |
| Cancer, No. (%)^{a} | 130 (42.1) |
| PHTS component malignant neoplasms, No. | |
| (%) | |
| Breast cancer | 58 (18.8) |
| Thyroid cancer | 36 (11.7) |
| Renal cell cancer | 25 (8.1) |
| Endometrial cancer | 27 (8.7) |
| Colon cancer | 10 (3.2) |
| Melanoma | 10 (3.2) |
| Nonmalignant features, No. (%)^{b} | 78 (25.2) |
| Macrocephaly | 70 (22.7) |
| Dermatologic features' | 42 (13.6) |
| Arteriovenous malformations | 10 (3.2) |
| Hemangiomas | 13 (4.2) |
| Polyposis | 38 (12.3) |
| Benign breast features^{d} | 21 (6.8) |
| Benign thyroid features^{e} | 49 (15.9) |
| Lhermitte-Duclos disease | 9 (2.9) |

Abbreviation: PHTS, PTEN hamartoma tumor syndrome
^{a}Includes 9 patients with neurodevelopmental disorders who have also been diagnosed with cancer
^{b}Includes patients without a personal history of neurodevelopmental disorders or cancer at the tissue of the last clinical visit and/or follow-up.
^{c}Includes trichilemmoma, acral keratosis, papillomatous papules, and genital lentiginosis (penile freckling in males).
^{d}Includes breast fibroadenoma, fibrocystic breast disease, breast papilloma, breast hamartoma, atypical ductal hyperplasia, and typical ductal hyperplasia.
^{e}Includes thyroid nodules, goiter, and Hashimoto thyroiditis.

Clinically Relevant CNVs in Patients with PHTS

The inventors identified 3 of 309 patients with PHTS (1.07%) and pathogenic and/or likely pathogenic CNVs affecting genes associated with PTEN-related cancers or other inherited cancer syndromes according to the American College of Medical Genetics and Genomics guidelines. These included 2 BMPR1A (OMIM 601299) deletions and 1 BRCA1 (OMIM 113705) deletion. The inventors could not ascertain cancer diagnoses because all 3 CNV carriers were children or young adults (aged 3, 6, and 21 years) without a personal history of cancer at the time of consent. The 2 patients with PHTS and BMPR1A deletions upstream of their individual PTEN deletion had juvenile polyposis, as is often observed in chromosome 10q23 microdeletions involving both genes (OMIM 612242). Notably, no pathogenic and/or likely pathogenic CNVs involving known cancer-associated genes were identified among patients in the subset with cancer.

Pathogenic and/or likely pathogenic CNVs involving genes implicated in the etiology of idiopathic non-syndromic and syndromic ASD/developmental delay and/or neurodevelopmental disorders as well as known pathogenic CNVs associated with ASD, developmental delay, and neurodevelopmental disorders were then investigated. The inventors found 11 of 110 patients in the ASD/DD group (10.0%) harbored such pathogenic and/or likely pathogenic CNVs, compared with 5 of 194 patients in the no ASD/DD group (2.6%) (OR, 4.2; 95% CI, 1.4-13.7; P=0.008) and 2 of 121 (1.7%) patients in the subset with cancer (OR, 6.6; 95% CI, 1.6-44.5; P=0.007) (FIG. 2). The CNV carriers included 10 male and 6 female patients with PHTS (mean [SD] age at consent, 22 [22.1] years). The CNVs included genomic loci associated with other genomic syndromes with overlapping clinical features, such as chromosome 8p23.1 duplication syndrome and chromosome 1q21.1 thrombocytopenia-absent radius syndrome.

### DISCUSSION

The present study evaluated the association of CNVs with clinical outcomes, specifically cancer vs ASD/DD, in individuals with germline pathogenic PTEN mutations. The inventors found that patients with PHTS and ASD/DD have an overall increased genome-wide CNV burden per individual and an enrichment of clinically relevant CNVs when compared with the subset of patients with cancer and those without ASD/DD. These data provide proof that CNVs may act as genomic modifiers of disease risk in phenotypically heterogeneous hereditary disorders such as PHTS.

As many as 23% of patients with PHTS develop ASD/DD. Conversely, 2% to 5% of unselected patients with ASD/DD and as many as 50% of children with ASD and macrocephaly have been found to carry germline PTEN mutations. Hansen-Kiss et al., J Med Genet., 54(7):471-478 (2017). As such, PTEN itself is a syndromic autism gene with well-established roles in nervous system development and neuronal function. Tilot et al., Neurotherapeutics, 12(3):609-619 (2015). However, why more than 75% of PTEN mutation carriers do not develop ASD/DD remains a clinical conundrum. These findings suggest pathogenic and likely pathogenic CNVs are associated with ASD/DD risk regardless of the particular coexisting germline PTEN mutation. Autism spectrum disorder is believed to be a highly heritable disorder, with complex genetic and biological basis. Pathogenic CNVs have been identified in 8% to 21% of individuals with idiopathic ASD, with higher frequencies reported in more severe cases, including causing syndromic ASD. Schaefer et al., Genet Med., 15(5):399-407 (2013). Notably, particular pathogenic CNVs have also been identified in neurotypical control individuals, including unaffected family members, albeit with significantly lower frequencies. Heil K M, Schaaf C P., Curr Psychiatry Rep., 15(1):334 (2013). First, this observation corroborates the incomplete penetrance and extreme variability in expression of the associated CNVs, meaning that not all carriers will manifest the associated clinical features. Second, this observation supports the burgeoning hypothesis that ASD etiology and severity are modulated through additive effects of multiple genomic aberrations, the so-called 2-hit (or multiple-hit) model. Demily et al., J Autism Dev Disord., 48(8):2886-2889 (2018). These characteristics are highly relevant in the context of modifiers within an inherently predisposed genetic background. In the case of PHTS, the inventors found evidence for the association of pathogenic CNVs with the coexisting germline PTEN mutations to favor ASD/DD clinical outcomes.

For example, CNVs involving CYFIP1 were identified in 3 unrelated patients with PHTS in the ASD/DD group. CYFIP1 encodes cytoplasmic FMR1-interacting protein 1, a protein originally found to interact with fragile X mental retardation protein. Increased CYFIP1 dosage has been shown to cause aberrant neuronal cellular and dendritic morphologic features and to be associated with ASD in humans and murine model organisms. Noroozi et al., Metab Brain Dis., 33(4):1353-1358 (2018). Importantly, CYFIP1 expression was inversely correlated with PTEN expression levels, with concomitant dysregulation of mammalian target of rapamycin (mTOR) signaling, suggesting that such CNVs may represent a second hit for patients with PHTS already harboring deleterious PTEN mutations that upregulate AKT-mTOR signaling. In cases of CYFIP1 duplications, decreased PTEN protein levels could also affect wild-type PTEN. In the context of CYFIP1 deletion, as was observed for 1 of the 3 patients with PHTS and CNVs involving CYFIP1 in this study, increased levels of a mutant PTEN protein may result in dominant negative effects (Papa et al., Cell, 157(3):595-610 (2014)), inhibiting wild-type PTEN and hence exacerbating the condition.

Notwithstanding the association of CNVs with multiple neurodevelopmental disorders, CNVs have also been implicated in the etiology of neuropsychiatric diseases such as epilepsy, schizophrenia, and bipolar disorder. Merikangas et al., Trends Genet., 25(12):536-544 (2009). A broad spectrum of neuropsychiatric phenotypes has been observed in patients with PHTS ranging from normal development and intelligence to severely debilitating neurocognitive and motor aberrations, including generalized anxiety, adult-onset movement disorders, obsessive-compulsive disorder, and psychosis. Whether particular CNVs affect the spectrum or severity of such motor and/or neurocognitive disease manifestations in PHTS warrants further scrutiny in larger independent prospective series.

### CONCLUSIONS

The identification of testable clinically relevant genomic modifiers in PHTS is important for risk stratification and serves as proof of principle for other phenotypically variable disorders. Patients with PHTS and ASD/DD have an increased burden of pathogenic and/or likely pathogenic CNVs associated with neurodevelopmental disorders. Although CNV analysis is indicated as a first-tier clinical diagnostic test for patients with unexplained ASD/DD, it is not standard to test for such genomic alterations in the setting of an established diagnosis with a syndromic high-penetrance mendelian gene such as PTEN. Copy number variation analysis on immediate identification of germline PTEN mutation in infants and children may be beneficial. The presence of pathogenic and/or likely pathogenic CNVs in PHTS would suggest high likelihood of ASD/neurodevelopmental delay, leading to timely referral to formal evaluation and, if the results are positive, subsequent therapy. The earlier such interventions are instituted, the better the outcomes

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of providing a diagnosis for a subject having Cowden-like syndrome or PTEN germline mutations, the method comprising the steps of:
(a) obtaining a biological sample from a subject;
(b) conducting a germline PTEN mutation and deletion analysis of genomic DNA from the biological sample;
(c) determining the level of copy number variation in the genomic DNA;
(d) comparing the level of copy number variation in the genomic DNA to a control value for copy number variation; and
(e) diagnosing the subject as having an increased risk of developing a neurodevelopmental disorder if the copy number variation level is higher than the control value.

2. The method of claim 1, wherein the biological sample is blood.

3. The method of claim 1, wherein the neurodevelopmental disorder is autism spectrum disorder.

4. The method of claim 1, wherein genomic DNA comprises the PTEN promoter region.

5. The method of claim 1, wherein the deletion analysis is conducted using a multiplex ligation-dependent probe amplification assay.

6. The method of claim 1, wherein the mutation analysis comprises denaturing gradient gel electrophoresis, high-resolution melting curve analysis, and directed Sanger sequencing.

7. The method of claim 1, wherein the step of determining the level of copy number variation comprises single-nucleotide polymorphism genotype quality control.

8. The method of claim 1, wherein the subject is human.

9. The method of claim 1, wherein the subject has Cowden-like syndrome.

10. The method of claim 1, wherein the subject has germline PTEN mutations.

11. The method of claim 10, wherein the germline PTEN mutations are pathogenic germline PTEN mutations.

12. The method of claim 1, wherein a copy number variation level that is higher than the control value indicates a higher risk of developing a neurodevelopmental disorder than the risk of developing cancer.
